(19) United States
(12) Patent Application Publication
(10) Pub. No.: US 2018/0147378 A1
(43) Pub. Date: May 31, 2018

(54) APPARATUS FOR ARTIFICIAL VENTILATION, WITH MONITORING FOR ABSENCE OF CHEST CONTRACTIONS

(71) Applicant: Air Liquide Medical Systems, Antony (FR)

(72) Inventors: Eric JACQUOT, Antony (FR); Thomas PENNORS, Antony (FR); Marceau RIGOLLOT, Montrouge (FR); Jean-Christophe RICHARD, Antony (FR)

(21) Appl. No.: 15/579,433

(22) PCT Filed: Apr. 28, 2016
(86) PCT No.: PCT/FR2016/051000

§ 371 Date: Dec. 4, 2017

(30) Foreign Application Priority Data
Jun. 8, 2015 (FR) 1555191

(51) Int. Cl. A61M 16/00; A61B 5/08; A61H 31/00; A61M 16/20

(52) U.S. Cl. CPC A61M 16/024; A61B 5/08; A61H 31/005; A61M 16/0069; A61M 16/205; A61H 31/007; G09B 23/288; A61M 2016/0027; A61H 2201/0184; A61M 2016/0021; A61M 2205/502; A61H 2201/5071; A61H 2230/42; A61H 2201/5007

(57) ABSTRACT

A respiratory assistance apparatus (1) for delivering a flow of gas to a patient (P) comprises a gas delivery conduit (2) for conveying a flow of gas, measuring means (6) designed to measure at least one parameter representative of the flow of gas and to supply at least one signal corresponding to said parameter, signal-processing means (8) designed to process said signal coming from the measuring means and to deduce from said signal at least one item of information relating to performance and/or discontinued performance of chest contractions (CT), calculating means designed to calculate at least one duration of discontinuation or absence of chest contractions (tNCT), and storage means (12) configured to register said duration of discontinuation or absence of chest contractions.

### CROSS REFERENCE TO RELATED APPLICATIONS

This application is a 371 of International PCT Application PCT/FR2016/051000, filed Apr. 28, 2016, which claims priority to French Patent Application No. 1555191 filed Jun. 8, 2015, the entire contents of which are incorporated herein by reference.

### BACKGROUND

The invention relates to an artificial ventilation apparatus that can be used during a cardiac massage on a ventilated patient, in order to ensure monitoring of the cardiac massage performed by a first aid provider, such as an emergency physician, a firefighter, a nurse or the like, in particular monitoring of the time without compression, and it relates to a monitoring method used by such an apparatus.

In the event of a cardiac arrest, when cardiopulmonary resuscitation (CPR) is performed, first aid providers must endeavor to ensure the fewest possible interruptions of the chest compressions, so as never to stop the cardiac output generated by the chest compressions, and so as not to drain the cardiac pump.

The time referred to as the no-flow time, that is to say the time during which the patient is without cardiac output, is a strong prognostic indicator for the survival of the patient without sequalae.

It is necessary to distinguish between the initial no-flow (or tNFᵢₙᵢₜ), corresponding to the time (i.e. duration) that elapses between the onset of the cardiac arrest and the first chest compressions, and the total no-flow (or tNFₜₒₜ), which also comprises all the interruptions of chest compressions (CT) during the management which is provided by the emergency services, that is to say by a first aid provider, such as a firefighter, an emergency physician or the like, and which is also referred to as specialized cardiopulmonary resuscitation. Each individual time of interruption of CT is called the “no-CT time” (or tNCT).

This is expressed by the following equation: tNFₜₒₜ=tNFᵢₙᵢₜ+S(tNCT)

where: S(tNCT) is the sum of all the times of interruption of the chest compressions.

Moreover, given that a specialized cardiopulmonary resuscitation phase systematically requires the implementation of artificial ventilation, in most cases performed by a respiratory assistance apparatus delivering a respiratory gas such as air to the patient via a patient interface such as a mask or an intubation cannula, the occasions on which the cardiac massage is interrupted are therefore numerous, in particular intubating the patient, analyzing the rhythm of the defibrillator, moving the patient, etc.

All of these interruptions, being brief and also numerous, are difficult for the medical personnel to quantify. However, they compromise the chances of survival and are therefore important to identify.

It will be readily appreciated that a first-aider providing first aid to a person in cardiac arrest does not have the possibility of keeping precise count of all the periods during which chest compressions have not been performed, since he or she is busy saving this person's life by administering the chest compressions.

The problem therefore is to be able to identify the total time (Ttot) without chest compression (CT), that is to say the total duration “no-CT”, of a person, i.e. a patient, who is in cardiac arrest and is ventilated by a respiratory assistance apparatus during CPR.

### SUMMARY

The solution of the invention thus concerns a respiratory assistance apparatus, that is to say a medical ventilator, for delivering a flow of gas to a patient, comprising:
a gas delivery conduit for conveying a flow of gas, in particular of air,
measuring means designed to measure at least one parameter representative of the flow of gas and to supply at least one signal corresponding to said at least one parameter representative of said flow of gas,
signal-processing means designed to process said at least one signal coming from the measuring means and to deduce from said signal at least one item of information relating to performance and/or discontinued performance of chest contractions (CT),
calculating means configured to calculate at least one duration of discontinuation or absence of chest contractions (tNCT) during which at least one item of information relating to a discontinuation or an absence of chest contractions (CT) is determined by the signal-processing means, and
storage means designed to register said at least one duration of discontinuation or absence of chest contractions (tNCT) calculated by the calculating means.

Depending on the circumstances, the respiratory assistance apparatus or medical ventilator of the invention can comprise one or more of the following technical features:
the measuring means are designed to measure at least one parameter representative of the flow of gas, chosen from among the gas pressure (P) and the gas flowrate (Q), in particular a flowrate of gas insufflated to the patient or a flowrate of gas exhaled by the patient.
the measuring means comprise at least one gas pressure sensor or a gas flowrate sensor.
the calculating means are configured to calculate several successive durations S(tNCT) of discontinuation or absence of chest contractions (tNCT) during which a discontinuation or an absence of chest contractions is determined. To put it another way, the calculating means calculate the sum S(tNCT) of the successive durations of discontinuation or absence of chest contractions (tNCT), by which means it is possible to obtain a total duration (Ttot) of absence of chest compressions.
the calculating means( ) are configured to determine the total duration (Ttot) of absence of chest compressions by adding together S(tNCT) the durations of discontinuation or absence of chest contractions (tNCT) during which a discontinuation or an absence of contractions is determined, that is to say the sum S(tNCT) of the different times without contraction (tNCT) is calculated.
the measuring means and the calculating means are configured to operate continuously in such a way as to detect and calculate continuously any discontinuation or absence of chest contractions.
it comprises display means designed to display at least one duration of discontinuation or absence of chest contractions (tNCT) or the total duration (Ttot) of discontinuation of absence of chest contractions.
the signal-processing means comprise at least one electronic board, preferably an electronic board comprising at least one microprocessor implementing at least one algorithm.
an (or the) electronic board comprises the calculating means.
the calculating means, also called “calculating device” or “calculator”, comprise a timer.
the display means comprise a man-machine interface.
the display means comprise a screen for viewing information, preferably a touch screen and/or color screen.
the calculating means are configured to increment and/or stop a calculator permitting measurement of the total time (Ttot) without chest contraction on the basis of all the determined durations without contraction (tNCT), during a cardiac massage.
the calculating means are configured to stop incrementing a calculator after definitive termination of the cardiac massage on a patient.
it comprises regulation or selection means designed to allow the user, that is to say the first aider or similar, to enter or select data.
the regulation or selection means are designed to provide information on a duration of initial no-flow (or tNFᵢₙᵢₜ) corresponding to the time (i.e. the duration) that has elapsed between the onset of the cardiac arrest of the patient and the first chest compressions performed on said patient, for example by a first aider or by a witness present at the scene. The duration of initial no-flow (or tNFᵢₙᵢₜ) can be an approximate time.
the regulation or selection means are designed to interact with, that is to say instruct, the ventilator and to modify the parameters of the ventilation delivered, or to enter or select data.
the regulation or selection means are designed to inform the ventilator of the commencement of cardiac massage.
the calculating means are configured to start incrementing the calculator permitting measurement of the total time (Ttot) without chest contraction on the basis of all the determined durations without contraction (tNCT), during a cardiac massage, in response to an indication by the user of a commencement of cardiac massage through activation of the regulation or selection means.
the man-machine interface comprises the regulation or selection means, for example one or more activation or selection buttons, one or more rotary knobs, one or more sliding cursors, or similar.
the man-machine interface comprises a touch screen comprising the duration input means, in particular touch buttons.
the calculating means are configured to add the duration of initial no-flow (tNFᵢₙᵢₜ) to the total time (Ttot) without chest contraction during the cardiac massage and to deduce therefrom the duration of total no-flow (tNFₜₒₜ) in the patient.
it comprises storage means designed to store information and/or data.
the storage means comprise at least one data storage memory, in particular a flash memory or the like.
the storage means configured to store one or more ventilation modes such as one or more modes of volumetric ventilation (VAC), barometric ventilation (VPC, VSAI, CPAP, Duo-Levels) and/or intermittent ventilation (VACI, PVACI).
the signal-processing means are configured to operate continuously and to ensure detection of chest contractions corresponding to an item of information of performance or non-performance of cardiac massage, and to deliver said information to the calculating means in such a way as to increment or not increment the calculator depending on the presence or the absence of chest contractions, that is to say of chest massage.
the signal-processing means and/or the calculating means comprise at least one electronic board, preferably a (micro)controller implementing one or more algorithms.
the man-machine interface is able and designed to display items of information including at least one item of information relating to the duration without cardiac massage comprising the duration of initial no-flow (tNFᵢₙᵢₜ), the total time (Ttot) without chest contraction during the cardiac massage and/or the duration of total no-flow (tNFₜₒₜ) in the patient.
it comprises reinitializing means which are configured to initialize or reinitialize the calculator, that is to say set it or reset it to 000 (i.e. zero).
the reinitializing means are configured to initialize or reinitialize the calculator after the user has acted on the regulation or selection means in order to inform the ventilator of commencement of cardiac massage. In other words, the calculator of the duration “tNCT” is initialized at 000, at the start-up of the ventilator, that is to say upon an action of the user indicating a change of patient.
it comprises means for producing a report at the end of the cardiac massage intervention, that is to say an account or the like including one or more items of information chosen from among the duration of performance of the cardiac massage, the total duration (Ttot) of absence of chest compressions, the duration of initial no flow (tNFᵢₙᵢₜ) and/or the duration of total no flow (tNFₜₒₜ).

### BRIEF DESCRIPTION OF THE DRAWINGS

The invention will now be better understood from the following detailed description which is given as a non-limiting example and in which reference is made to the accompanying figures, in which:

FIG. 1 shows an embodiment or a respiratory assistance apparatus according to the present invention, in which the gas source is a micro-blower, and

FIG. 2 is a graph illustrating the pressure and the flowrate of the gas insufflated into a patient, showing the periods with and without chest compressions, and operations effected by the ventilator according to the invention.

### DESCRIPTION OF PREFERRED EMBODIMENTS

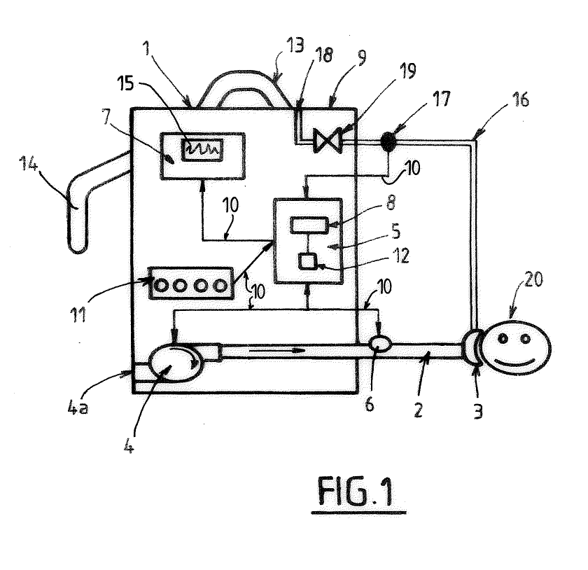

FIG. 1 is a schematic representation of an embodiment of a ventilatory assistance apparatus or medical ventilator 1 according to the present invention.

It comprises a gas source 4, which is here a motorized micro-blower, also called a turbine, delivering a flow of respiratory assistance gas, typically a flow of air or of oxygen-enriched air. The air is aspirated by the micro-blower via one or more inlet orifices 4a formed in the shell 9 of the ventilator 1.

In an alternative (not shown), the gas source 4 comprise a controlled valve supplied with gas via an internal conduit, which is itself in fluidic communication with a gas reservoir or a gas supply wall socket connected to a gas mains network by way of a flexible conduit connecting the gas reservoir or the wall socket to the internal conduit.

The ventilatory circuit 2, 16, also called the patient circuit, comprising one or more passages, conduits or gas lines, makes it possible to fluidically connect the gas source 4 of the ventilator 1 to the airways of a patient 20, by way of a patient interface 3, such as a breathing mask or an intubation cannula.

The ventilatory circuit 2, 16 comprises at least one inhalation branch 2 for conveying the respiratory gas to the patient 20. It can also comprise an exhalation branch 16 designed to collect the gases exhaled by the patient 20, which are rich in CO₂.

The exhalation branch 16 comprises an exhalation flowrate sensor 17, for example a hot-wire sensor, connected electrically to the control means 5, such as an electronic board, and also an exhalation valve 19 controlled by the control means 5. At its downstream end, the exhalation branch 16 communicates with the atmosphere via a gas outlet orifice 18, while its upstream end is connected to the inhalation branch 2, via a Y-shaped piece, or directly to the patient interface 3.

Measuring means 6 are provided, such as a sensor and/or flowrate, which are able and designed to measure at least one parameter representative of the flow of gas, in particular the gas pressure or the gas flowrate, whether the flowrate insufflated by the respirator and/or the flowrate of gas exhaled by the patient 20, and to deliver at least one signal representative of said at least one measured parameter.

The measurement is effected in the inhalation branch 2 of the ventilatory gas circuit 2, 16 in such a way as to permit a measurement of the pressure or the flowrate of gas in said inhalation branch 2 serving as gas delivery conduit. In the embodiment illustrated in FIG. 1, the measuring means 6 are arranged outside the ventilator. However, they can also be situated within the ventilator 1, according to another embodiment.

Once the one or more parameters representative of the flow of gas have been measured, this measured parameter is converted into at least one signal representative of the flow of gas, which signal is then transmitted to and analyzed by signal-processing means 8, that is to say typically the electronic board serving as control means 5, in order to deduce from said signal at least one item of information relating to a cardiac massage performed on a patient who is in cardiac arrest, in particular at least one item of information relating to performance and/or discontinued performance of chest contractions CT.

The signal-processing means 8 thus form part of the control means 5 of the ventilator 1 and comprise one or more electronic boards with microprocessor(s) implementing one or more algorithms.

The ventilator 1 additionally comprises calculating means, for example a counter or a timer, which cooperate with the signal-processing means 8.

More precisely, the calculating means are configured, that is to say designed and able, to calculate each duration of discontinuation or absence of chest contractions (tNCT) in response to the detection of an item of information relating to a discontinuation or absence of chest contractions (CT) by the signal-processing means 8.

All the successive durations tNCT of discontinuation or absence of chest contractions are calculated and added up to obtain a total duration Ttot of absence of chest compressions.

Storage means 12, such as a flash memory, permit registration of the one or more durations of discontinuation or absence of chest contractions (tNCT) calculated by the calculating means, and also the total duration Ttot of absence of chest compressions.

The ventilator 1 additionally comprises a man-machine interface comprising one or more control buttons 11 that can be activated by the users, that is to say the medical personnel, for example a first-aid worker, and also comprising an information display screen 7, said man-machine interface preferably comprising a touch screen with color display, of which certain zones constitute control keys.

When the signal-processing means 8 have processed the one or more signals coming from the measuring means 6 and have deduced therefrom an item of information characteristics of a cardiac massage performed on the patient 20, the calculating means then start counting the different durations tNCT of discontinuation or absence of chest contractions during which a discontinuation or an absence of contractions is determined, that is to say they start adding these durations together to be able to deduce from them a total duration Ttot of absence of chest compressions.

Preferably, the measuring means 6 and the calculating means are configured to operate continuously in such a way as to continuously detect any discontinuation or absence of chest contractions, and any resumption of cardiac massage.

Preferably, the calculating means are configured to start incrementing the counter permitting measurement of the total time Ttot without chest contraction on the basis of all the durations without contraction tNCT determined, during a cardiac massage, in response to an indication by the user of an initiation of a ventilation mode specific to a cardiopulmonary resuscitation, by activation of regulation or selection means, for example after the user has pressed a button indicating a commencement of cardiac massage.

Finally, the ventilator 1 displays, on the man-machine interface, a duration tNCT of discontinuation or absence of chest contractions or the total duration (Ttot) of discontinuation or absence of chest contractions corresponding to the sum S(tNCT) of all the durations tNCT of discontinuation or absence of chest contractions.

Means for producing a report are provided, such as display of the report at the end of the intervention or the possibility of retrieving a report at the end of the intervention on a USB storage device or similar. They are preferably designed to produce a report of the intervention after the cardiac massage, that is to say an account or the like including one or more items of information chosen from among the duration of performance of the cardiac massage, the total duration Ttot of absence of chest compressions, the duration tNFᵢₙᵢₜ of initial “no flow” and/or the duration tNFₜₒₜ of total “no flow”.

The signals are transmitted between the different components of the ventilator 1 via suitable links, that is to say electrical links 10 such as cables or the like.

The ventilator 1 can also comprise a carrying handle 13 and/or a securing system 14 such as a hook or the like serving to secure the apparatus to a support. The ventilator 1 can also be transported in a rigid case or a flexible bag.

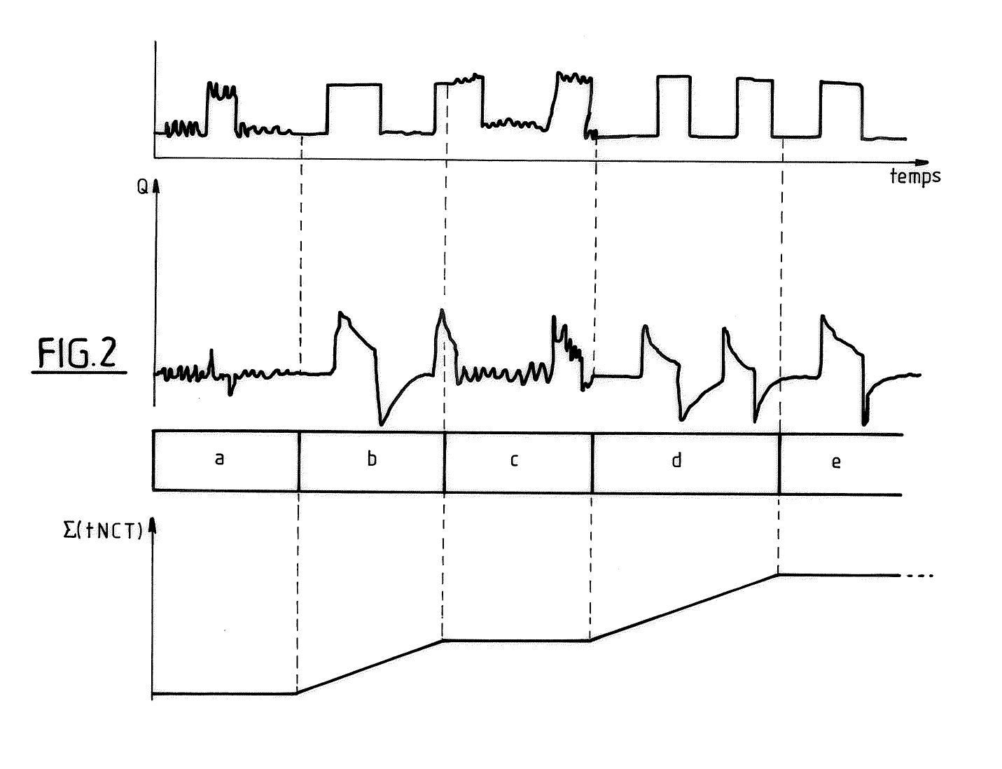

FIG. 2 is a graph illustrating the pressure P and the flowrate Q of the flow of gas insufflated to a patient over the time t, by means of a ventilator according to the invention, for example the ventilator of FIG. 1, from which it is possible to distinguish the periods with (phases a and c) and without (phase b) chest compressions and also a representation of the operations effected by the respirator.

More precisely, when the respirator 1 detects the presence of a cardiac massage (phases a and c) by analyzing the signal of flowrate Q and/or pressure P coming from the used flowrate or pressure sensor 6, the duration tNCT that has elapsed without chest compressions is not incremented.

Conversely, when the respirator does not detect cardiac massage (phase b) on analyzing the signal of flowrate Q and/or of pressure P, the duration tNCT that has elapsed without chest compression is incremented, that is to say it is added to the other durations tNCT which have been able to be detected previously and added to one another to determine the total duration Ttot reflecting the absence of chest compressions CT.

In the case of a prolonged period (1 minute for example) without chest compressions (phase d), the ventilator considers that the patient has been resuscitated or that the attempt at resuscitation has been abandoned. In this phase c, the counter tNCT is no longer incremented.

Knowledge of tNCT will make it possible to calculate and display the percentage of the resuscitation time during which compressions are performed, in such a way as to encourage the medical personnel never to interrupt the chest compressions for approaching the ideal value of 100%. This index, which we will call % CT, is calculated as follows:

$$\% CT = \frac{tNCT}{Ttot} * 100$$

Thereafter, the duration of performance of the cardiac massage, the total duration Ttot of absence of chest compressions, the duration tNFᵢₙᵢₜ of initial “no flow”, and/or the duration of tNFₜₒₜ of total “no flow” can be displayed on the touch screen 7 of the ventilator 1 and/or can be the subject of an intervention report intended for the medical personnel.

Generally, the artificial ventilation apparatus of the invention is perfectly adapted for use during a cardiac massage on a ventilated patient, in order to ensure monitoring of the cardiac massage performed by the first aid provider, such as an emergency physician, a firefighter, a nurse or the like.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A respiratory assistance apparatus (1) for delivering a flow of gas to a patient (P), comprising:
a gas delivery conduit (2) adapted to deliver a flow of gas,
measuring device (6) designed to measure at least one parameter representative of the flow of gas and to supply at least one signal corresponding to said at least one parameter representative of said flow of gas,
a signal-processing system (8) designed to process said at least one signal coming from the measuring device (6) and to calculate from said signal at least one item of information relating to a performance and/or a discontinued performance of chest contractions (CT),
the signal-processing system (8) further designed to calculate at least one duration of the discontinuation or an absence of chest contractions (tNCT) during which at least one item of information relating to the discontinuation or the absence of chest contractions (CT) is determined by the signal-processing system (8), and
a storage device (12) designed to register said at least one duration of discontinuation or absence of chest contractions (tNCT) calculated by the calculating means.

2. The apparatus of claim 1, wherein the at least one parameter representative of the flow of gas is chosen from among the gas pressure (P) and the gas flowrate (Q), and wherein the measuring device (6) comprises at least one gas pressure sensor or a gas flowrate sensor.

3. The apparatus of claim 1, wherein the signal-processing system (8) is configured to calculate several durations of discontinuation or absence of chest contractions (tNCT) during which a discontinuation or an absence of contractions is determined.

4. The apparatus of claim 1, wherein the signal-processing system (8) is configured to determine a total duration (Ttot) of absence of chest compressions by adding together durations of discontinuation or absence of chest contractions (tNCT) during which the discontinuation or the absence of contractions is determined.

5. The apparatus of claim 1, wherein the measuring device (6) and the signal-processing system (8) are configured to operate continuously in such a way as to detect and calculate continuously any discontinuation or absence of chest contractions.

6. The apparatus of claim 1, further comprising a display designed to display at least one duration of discontinuation or absence of chest contractions (tNCT) or a total duration (Ttot) of discontinuation or absence of chest contractions.

7. The apparatus of claim 1, wherein the signal-processing system (8) comprises at least one electronic board comprising at least one microprocessor configured to implement at least one algorithm.

8. The apparatus of claim 1, wherein the display (7) comprises a screen for viewing information.

9. The apparatus of claim 1, wherein the signal-processing system (8) is configured to start incrementing the calculator permitting measurement of the total time (Ttot) without chest contraction on the basis of all the determined durations without contraction (tNCT), during a cardiac massage, in response to an indication by a user of a commencement of cardiac massage through activation of a regulation or selection device.

10. The apparatus of claim 1, wherein the signal-processing system (8) comprises a counter or a timer.

11. The apparatus of claim 1, wherein the electronic board comprises the signal-processing system (8).
